(19) United States
(12) Patent Application Publication
(10) Pub. No.: US 2006/0167419 A1
(43) Pub. Date: Jul. 27, 2006

(54) Device for administering measured doses of a liquid product with a dockable display

(76) Inventors: Patrick Fiechter, Sumiswald (CH); Christof Meier, Utzenstorf (CH); Philippe Kohlbrenner, Kaltacker (CH); Urs Widmer, Bern (CH)

Correspondence Address: DORSEY & WHITNEY LLP;INTELLECTUAL PROPERTY DEPARTMENT, 50 SOUTH SIXTH STREET, MINNEAPOLIS, MN, 55402-1498, US

(21) Appl. No.: 11/316,422

(22) Filed: Dec. 22, 2005

(30) Foreign Application Priority Data
Dec. 31, 2004 (DE) 10 2004 063 649.4

(51) Int. Cl. A61M 5/00

(52) U.S. Cl. 604/181

(57) ABSTRACT

A device for administering measured doses of a liquid product with a dockable dose display (i.e., a dose display that can be connected, attached, operably coupled to, fitted or retrofit on the device after the device is manufactured), the device including a housing with a reservoir for the product, a plunger rod displaceable to convey the product in a forward drive direction, a dose-measuring member for making a dose-measuring movement to set a product dose to be administered, a dose display with at least a first display element for displaying the set dose, and a coupling which couples the dose display with the dose-measuring member to record, track and/or display a dose-measuring movement, wherein the dose display comprises an interface coupled with the dose-measuring member for communicating the dose measuring to a user.

### CROSS-REFERENCE TO RELATED APPLICATION(S)

This application claims the benefit of German Application No. DE 10 2004 063 649.9, filed on Dec. 31, 2004, the contents of which are incorporated in its entirety herein by reference.

### BACKGROUND

The invention relates to devices for administering, dispensing or delivering substances and methods of making and using such devices. More particularly, it relates to medical devices, including devices for administering measured doses of a liquid product. The device may be an injection device, including an injection device in the form of an injection pen.

Injection devices are known, including from their use or applications involving the treatment of diabetes and the administration of growth hormones or osteoporosis preparations. On the one hand, devices of this type should offer some assurance that the correct dose will be administered and, on the other hand, the devices should be easy and comfortable to use, two requirements which are of particular importance if the user is self-administering the relevant product.

Injection devices, including injection pens, designed for self-administering settable doses of a product to be injected are known. For example, U.S. Pat. No. 6,547,763 describes a dose display integrated in such an injection pen, comprising two flexible discs, each of which has a scale. In the middle of the discs is a circular orifice, by means of which they can be mounted in an injection pen so as to be freely displaceable. On a radius that is bigger than that of the orifice but smaller than that of the disc, a larger number of small orifices or perforations is provided. In the assembled state, cams locate in these perforations, which are borne by means of a cylindrical body coaxially integrated in an injection device and connected to a dose-measuring element so as to be prevented from rotating. A rotation on the dose-measuring element transmits this rotating motion to the flexible discs.

### SUMMARY

An object of the present invention is to provide a dose display for injection devices, which can be fitted onto pre-fabricated injection devices at a date later in time than that on which the devices were manufactured, for example, on the delivery date of the devices or before the delivery of the devices to care-givers or patients.

In one embodiment, the present invention comprises a dose display for coupling to an injection device or the like, comprising at least one display element, a gear mechanism and a central element which connects the display element, the gear mechanism and the injection device whereby the dose display can be coupled as a unit to appropriately prepared injection devices after the injection devices are manufactured.

In one embodiment, the invention comprises an injection device comprising a dose display comprising at least one display element, a gear mechanism and a central element which connects the display element, the gear mechanism and the injection device whereby the dose display can be coupled as a unit to the injection device after the injection device has been manufactured.

In one embodiment, the invention relates to a dose display of compact construction, which can be laterally docked. It comprises at least one, but in some embodiments, preferably and advantageously precisely two display elements, for example counter dials, a gear mechanism and a central element which connects all of the components of the dose display in such a way that they do not have to be fitted or retrofit as a functional unit to appropriately prepared devices until shortly before delivery to the customer. Docking, fitting and the like are intended to encompass connecting, attaching, mounting, operably coupling, retrofitting, mounting, linking, etc.

In one embodiment, the present invention comprises a device for administering measured doses of a liquid product with a dockable dose display (i.e., a dose display that can be connected, attached, operably coupled to, fitted or retrofit on the device after the device is manufactured), the device including a housing with a reservoir for the product, a plunger rod displaceable to convey the product in a forward drive direction, a dose-measuring member for making a dose-measuring movement to set a product dose to be administered, a dose display with at least a first display element for displaying the set dose, and a coupling which couples the dose display with the dose-measuring member to record, track and/or display a dose-measuring movement, wherein the dose display comprises an interface coupled with the dose-measuring member for communicating the dose measuring to a user.

Although a drive member of a device may simultaneously also serve as a dose-measuring member of the device on which the user sets the dose, in some preferred embodiments a device in accordance with the present invention has a dose-measuring member in addition to the drive member. The dose-measuring member is coupled with the drive member, preferably by purely mechanical means, so that a dose-measuring movement of the dose-measuring member also results in a dose-measuring movement of the drive member. The dose-measuring movement of the drive member is effected in the direction opposite the driving force applied by or via the drive member during the dispensing process. The device has a dose display of the type provided by the present invention for displaying the set product dose. The display may be of the acoustic, tactile and/or visual type. The dose display is coupled with the dose-measuring member so that a movement effected by the dose-measuring member when a product dose is set causes a change in the displayed product dose. When the coupling members are in the retaining position, the dose-measuring member and/or the dose display are uncoupled from the conveying mechanism. As a result of the uncoupling, the dose can be set and corrected if necessary in the uncoupled state, without affecting the conveying mechanism.

In a preferred embodiment, the coupling between the dose display and drive member stays in coupled engagement so that during progressive dispensing, a driving movement of the drive member effected in the direction opposite the dose-measuring movement is likewise progressively covered. If the administration process is prematurely interrupted, whether deliberately or in error, the dose display therefore shows the remaining quantity of the set dose that has not yet been dispensed. This may be of advantage if the set dose is bigger than the amount still available.

If, as in preferred embodiments, a dose-measuring member is provided in addition to the drive member, the drive member and the dose-measuring member are advantageously uncoupled from one another when the coupling engagement is established, so that the dose-measuring member can not be manipulated in any way which might affect the drive member as the drive member is being moved.

In another embodiment, when the coupling members are in the retaining position, the coupling output member is secured on the housing so that it is not able to make any movement which might affect a dispensing movement of the conveying mechanism. The coupling output member must be deliberately released, preferably directly in conjunction with dispensing the product. It is of advantage if the connection to the housing part is released as the coupling motion is effected. The coupling engagement is produced during a first portion of travel covered during the coupling movement in a first phase, and the connection on the housing is then released in a subsequent second phase, advantageously against the elastic rebound force of the retaining device. The coupling output member is advantageously fixed on the housing by means of the coupling interconnecting member when the coupling members are in the retaining position. The locking engagement of the coupling interconnecting member with respect to the housing or a structure connected to it for this purpose is expediently released when the coupling movement is effected. It is of advantage if the coupling interconnecting member can be moved out of the locking engagement in the direction of the coupling movement because such a movement enables the coupling interconnecting member to be simply driven during the coupling movement, for example pushed out of the locking engagement. The locking engagement may be of the positive and/or friction type.

In a first variant, the return member acts on the at least one coupling member effecting the coupling movement via the coupling interconnecting member, retaining it in the retaining position. In a second variant, the return member acts directly on the at least one coupling member effecting the coupling movement, preferably the coupling input member, and is supported on the housing or a structure which is fixedly connected to the housing for the purpose of the coupling movement or to the coupling output member in order to apply pressure, for example.

In some embodiments, the coupling movement is preferably an axial stroke movement. If a plunger and a plunger rod constitute the conveying mechanism, the stroke movement is preferably effected in the forward-drive direction of the plunger and the plunger rod. As the driving force drives the coupling input member in rotation, which in turn drives the coupling output member in rotation due to the coupling engagement, advantageously about an axis of rotation pointing in the forward drive direction, those coupling members which between them form the coupling engagement are specifically provided with locating elements, which co-operate in the manner of axially, mutually displaceable grooves and springs or as teeth axially directed towards one another or preferably conical teeth. Although a single tooth and a single tooth gap would be sufficient to produce the coupling engagement in theory, it is preferable for at least one of the coupling members co-operating in the coupling engagement to have a set of teeth extending peripherally around the axis of rotation. Even more preferably, in some embodiments, both of the coupling members co-operating in the coupling engagement each have a set of circumferentially extending teeth. The same applies if the engaging elements are provided in the form of grooves and springs or some other means. Irrespective of what shape or form the coupling surfaces are, the coupling engagement is designed so that no slip occurs in the coupling engagement.

To render production of the injection devices as flexible as possible, it is advantageous to split the devices into modules, which are individually manufactured and stored and not assembled until packaging. The dose display may be one such module, in which case it is assembled with a prefabricated injection device shortly before being delivered to the customer. The dose display proposed by the invention is a dose display with a modular construction of this type.

The dose display consists of a first and an additional display element, a gear mechanism consisting of a display coupling member which establishes the connection to the device in the assembled state and simultaneously drives the first display element, a mechanism by means of which the first display element is able to drive an additional display element about a circle segment with every complete turn, and a centrally disposed sleeve serving as a connecting element for these individual components.

When setting the dose by turning a dose-measuring mechanism, for example, which in the case of self-injection devices is done manually by the user as a rule, the rotating movement of the drive member, which is connected to both the dose-measuring member and the dose display during the dose-measuring process, is deflected via the gear interface and transmitted to the dose display. The user continues to rotate the dose-measuring mechanism until a reading of the desired dose appears on the dose display. If a higher dose is unintentionally set, a correction can be made to the desired value by a simple backward turn on the dose-measuring member, which backward turn likewise causes the requisite correction to be made to the dispensed quantity. Instead of effecting a rotating movement on the dose-measuring device, it would also be possible to use a setting mechanism operated on the basis of an axial displacement or a permitted tipping movement of the dose-measuring device transversely to the device longitudinal axis. The dose measurement can likewise by operated by an electric, pneumatic or some other drive, in addition to which the forward drive of the dispensing mechanism need not be limited to a manual driving action and/or spring force.

When the injection is initiated, the dose display is connected via appropriate coupling elements to the drive mechanism causing the dispensing action and is driven by it during dispensing. Once the set dose has been fully administered, the dose display indicates the value zero on termination of the injection. If the injection process is inadvertently or deliberately interrupted as the product is being dispensed, the dose display indicates the quantity of product which still has to be dispensed at that instant. In this case, the user can set the display to zero or continue administering the product and thus administer the product quantity which has not yet been injected. If, during the injection, the user notices that he has set too low or too high a dose on the dose-measuring member, he can interrupt the injection at any time, correct the remaining quantity of product to be dispensed at this point in time and then finish the injection.

A real-time display proposed by the invention may be provided with mechanical elements of two or more mutually encompassing counter dials, which are printed with the figures “0” to “9” in the circumferential direction. A display of this type incorporating mutually encompassing counter dials may be provided with a setting knob or dose-measuring member, coupled so that whenever the setting knob or the dose-measuring member is moved, the mechanical display also turns during the process of measuring the dose and/or correcting the dose, for example.

The mechanical dose display proposed by the invention consisting of mutually encompassing counter dials, for example, may likewise be coupled with the dispensing mechanism by means of appropriate coupling members so that the counter dials also turn during the dispensing process but in the direction opposite the setting direction. In other words, the mechanical display counts back from the set dose to the value zero as the product contained in the injection device is being dispensed. If the injection is unintentionally or deliberately interrupted, the movement of the dose display also stops and the reading indicates the part-dose which has not as yet been dispensed.

The first display element is preferably sub-divided into ten equal parts and serves as a counter dial for indicating the single figure units. A disc with a cam is mounted on the base end of the first display element which turns with the first display element and turns the latter one circle segment further with every rotation by means of a capstan wheel connected to the second display element.

The other display element may serve as a counter dial for the tens and, like the first display element, is divided into ten segments. In this embodiment, the circle segment about which the first display element drives the second display element with each rotation is exactly 36°. This rotation on the other display element would take place during the switch of the first counter dial from the digit “9” to the digit “0”, for example. However, any other sub-division may be used for the other counter dial by designing the capstan wheel accordingly.

The counter dials may be totally or partially transparent. This being the case, at least one light source, preferably a LED or OLED may be provided as a fixed component of the device. In some embodiments, the light source is preferably mounted underneath the circle segment which indicates the actual dose. The light source may be permanently illuminated or switched on by a defined event, for example at the start of the dose-measuring movement on the dose-measuring member, and be switched back off again at the end of the dispensing process, i.e. on reaching the zero position. However, it would also be conceivable to opt for a system which has to be deliberately switched on by the user in darkness or poor light conditions, for example by means of a knob or any other appropriate means which might be used for switching purposes.

Another possible solution is to apply the digits to the counter dials with fluorescent paint, in which case stored luminescent energy will enable a reading to be taken from the dose display in darkness.

A dose display proposed by the invention may be integrated in both an injection pen for self-administration of a product and in a mechanical, pneumatic or electrical injection device. It is also possible to replace the mechanical dose display with an electronically generated display. To this end, the rotation of the dose-measuring member and the movement of the dispensing mechanism are detected by appropriate sensors. The information obtained in this manner is converted into a digital display in an appropriate manner, by suitable hardware and/or software. It is also possible to display a real-time reading of the product quantity waiting to be dispensed in any situation, as described above.

For the purpose of integrating it in prefabricated injection devices, the dose display has an interface, which ensures both a reliable coupling of the drive with the drive of the injection device but also simultaneously ensures that the dose display is securely attached to the device.

### BRIEF DESCRIPTION OF THE DRAWINGS

FIG. 1 is a perspective view showing an exemplary embodiment of an injection device in accordance with the present invention,

FIG. 2 is a longitudinal section of the injection device with an open coupling,

FIG. 3 shows the injection device with a closed coupling,

FIG. 4 shows a detail from FIG. 2,

FIG. 5 shows a detail from FIG. 3,

FIG. 6 shows the injection device after setting a dose,

FIG. 7 shows the injection device after emptying a reservoir,

FIG. 8 is a detailed diagram showing the dose display in cross-section,

FIG. 8a is a detailed diagram showing a plan view of the dose display,

FIG. 9 is a cross-section of the dose display with an alternative drive and a light element,

FIG. 9a is a plan view of the dose display with an alternative drive and a light element.

### DETAILED DESCRIPTION OF THE DRAWINGS

With regard to fastening, mounting, attaching or connecting the components of devices of the present invention, unless specifically described as otherwise, conventional fasteners such as screws, rivets, toggles, pins and the like may be used. Other fastening or attachment means appropriate for connecting components include friction fitting, adhesives, welding and soldering, the latter particularly with regard to electrical or processing components or systems of the devices. Any suitable mechanical or power communicating links, linkages or transmission may be used. Any suitable electronic, electrical, communication, computer or processing components may be used, including any suitable electrical components and circuitry, wires, wireless components, sensors, chips, boards, micro-processing or control system components, software, firmware, hardware, etc.

FIG. 1 illustrates an exemplary embodiment of an injection device in accordance with the present invention. The injection device has a first housing part 1 and a second housing part 4, which are releasably connected to one another. In the embodiment illustrated here, the housing parts 1 and 4 are screwed together. The injection device is provided in the form of a slim injection pen. Housing part 1 is used to accommodate a container 2 filled with liquid product and in this sense constitutes a reservoir, while housing part 4 is used as a support for a dose-measuring and drive unit, in which a dose-measuring member 18 may be seen. In the region of the dose-measuring member 18, the housing part 4 is open, giving a user direct access to the dose-measuring member 18. The dose-measuring member 18 is mounted so as to be rotatable about a central longitudinal axis of the device and is provided in the form of a sleeve, which is knurled on its external circumference to make it ergonomically, user friendly. As illustrated, a dose display 20 is provided, which is fitted from the side through an orifice in the casing of the housing part 4.

FIG. 2 is a longitudinal cross-sectional view of the injection device in accordance with the present invention. The container 2 is accommodated in housing part 1. Disposed in the container 2 is a plunger 3, which is displaceable in a forward drive direction V. The plunger seals off the container 2 in a liquid-tight arrangement at its proximal end. When the plunger 3 is driven forwards in the forward drive direction V, the product is forced through an outlet of the container 2 and dispensed, preferably through an injection needle projecting into the outlet and attached to the distal end of the housing part 1 by means of a needle holder. The container 2 is provided in the form of a standard ampoule. Housing part 1 directly forms a container holder, which is an ampoule holder in the embodiment illustrated as an example here. The housing part 1 projects into the housing part 4 by its proximal end and is screwed to housing part 4.

Housing part 4 accommodates a plunger rod 15 and the dose-measuring and drive unit, which constitutes the dose-measuring and drive mechanism. In a dose-measuring and drive train, the dose-measuring and drive unit has a drive member 5 and a coupling, which, in a coupled state, i.e., in a coupling engagement, joint or mesh, couples the drive member 5 with the plunger rod 15. The plunger rod 15 forms a conveyor unit in conjunction with the plunger 3. In the coupled state, the coupling members 6-10 transmit a driving force applied to the plunger rod 15 to the drive member 5. In FIG. 2, there is no coupling engagement and the plunger rod 15 is therefore uncoupled from the drive member 5. In this uncoupled state, the user is able to set the product dose to be administered by a dose-measuring movement of the dose-measuring member 18 which, in the example of the embodiment illustrated here, is a rotating movement.

The drive member 5 is sleeve-shaped. It has a thread on its external surface extending around a thread axis R pointing in the forward drive direction V. The drive member 5 sits in a threaded engagement with a coupling input member 6 by means of this thread. The coupling input member 6 is also sleeve-shaped and is provided with a matching internal thread in order to establish the threaded engagement. The thread pitch in the threaded engagement is big enough to prevent any sticking due to friction. The dose-measuring member 18 surrounds the coupling input member 6 and is secured to the coupling input member 6 so that it is fixed with it in rotation and is connected to it so that it can not be axially displaced. The plunger rod 15 projects into the drive member 5 and the coupling input member 6.

The plunger rod 15 is provided with an external thread across its axial length. By means of the external thread, it sits in a thread engagement with a coupling output member 9, which is provided with a matching internal thread. The pitch of these two threads is also such that it prevents any sticking due to friction. The thread pitch is preferably smaller than the thread pitch of the thread engagement between the drive member 5 and the coupling input member 6. A coupling sleeve 8 is secured to the coupling output member 9 so as to be fixed to it in rotation and can not be axially displaced. The coupling sleeve 8 and the coupling output member 9 may be regarded as an integral component in terms of the movements which can be effected between the drive member 5 and the plunger rod 15; however, in order to accommodate a compensating spring 17, they are effectively provided in two pieces and are fixedly connected to one another. The coupling output member 9 and the coupling sleeve 8 are able to rotate about the thread axis R of the coupling output member 9 but are mounted in the housing part 4 so that they can not be axially displaced. When the coupling output member 9 is engaged by the thread, the plunger rod 15 projects through the coupling sleeve 8. A compensating spring 17 is biased between a proximal end of the coupling sleeve 8 and a proximal end of the plunger rod 15, which acts on the plunger rod 15 as a compression spring in the forward drive direction V. The compensating spring 17 is pushed onto the plunger rod 15 by means of a plate 15a, rotatably supported on the plunger rod 15, which forms a flange of a sleeve placed on the plunger rod 15.

Disposed in a linear guide 4a, the plunger rod 15 is not able to rotate relative to the housing part 1 and is linearly guided in and opposite the forward drive direction V. The drive member 5 is also displaceable relative to the housing part 4 and is likewise linearly guided in and opposite the forward drive direction V, for which purpose the housing part 4 directly serves as a linear guide 4b.

The thread axis of the plunger rod 15 forms the main displacement axis of the device. It constitutes a rotation axis R for the rotating driving motion of the coupling input member 6 and, via the coupling interconnecting member 7, of the coupling output member 9 as well. It also constitutes the translation axis for the plunger rod 15 and the drive member 5.

The coupling additionally comprises a coupling interconnecting member 7 and a return member 10, provided in the form of a compression spring which biases the coupling interconnecting member 7 with an elastic force acting in the direction opposite the forward drive direction V. The return member 10 is biased between the coupling output member 9 and the coupling interconnecting member 7.

When no force is acting on the drive member 5 in the forward drive direction V, the return member 10 ensures that the coupling engagement is released via the coupling interconnecting member 7. This state is illustrated in FIG. 2. The coupling input member 6 is pushed in the forward drive direction V on a stop against the coupling interconnecting member 7 and is pushed by the return member 10 via the coupling interconnecting member 7 into a proximal end position. The return member 10 holds the coupling input member 6 relative to the coupling output member 9 and the coupling sleeve 8 secured to it in a holding position by means of the coupling interconnecting member 7. The return member 10 and the coupling interconnecting member 7 therefore constitute a positively acting holding unit for the coupling input member 6.

FIG. 3 illustrates the injection device in a coupled state. A coupling engagement exists between the coupling input member 6 and the coupling sleeve 8. To establish the coupling engagement, the coupling member 6 and the coupling sleeve 8 constitute engaging elements, which establish a connection between the two members 6 and 8, which are fixed in rotation about the thread axis R pointing in the forward drive direction V when the coupling is engaged. The engaging elements co-operate by means of grooves and springs or teeth, which are uniformly distributed about the thread axis R, parallel with the forward drive direction V.

FIGS. 4 and 5 provide a detailed illustration of the region of the coupling engagement. FIG. 4 shows the device in the uncoupled state and FIG. 5 shows the coupled state. FIG. 4 therefore corresponds to FIG. 2 and FIG. 5 corresponds to FIG. 3.

In the uncoupled state, the coupling input member 6 is pushed back away from the coupling sleeve 8 in the direction opposite the forward drive direction V so that the coupling input member 6 is freely rotatable relative to the coupling sleeve 8 and hence the coupling output member 9 fixedly connected to it. At the same time, the coupling output member 9 is rotationally fixed to the housing part 4 by means of the coupling sleeve 8, by means of the coupling interconnecting member 7 and an uncoupling member 11. To establish or create this rotationally fixed coupling, the coupling interconnecting member 7 is provided with engaging elements 7b on an internal surface directed radially towards the coupling sleeve 8 and the coupling sleeve 8 is provided with matching engaging elements 8b. To establish the rotationally fixed engagement with the uncoupling member 11, the coupling interconnecting member 7 is provided with engaging elements 7a on an external peripheral surface and the uncoupling member 11 is provided with radially directed engaging elements 11a on an internal surface, which engage in the same way as the engaging elements 7b and 8b in the uncoupled state in the form of grooves and springs or teeth parallel with the forward drive direction V, which locate in one another. In its rotationally fixed engagement with the coupling sleeve 8 and its rotationally fixed engagement with the uncoupling member 11, the coupling interconnecting member 7 is axially displaceable in and opposite the forward drive direction V and the engagement with the uncoupling member 11 is released when a displacement is effected in the forward drive direction V.

When the drive member 5 is operated by exerting a compression force on a release element 16 in the forward drive direction V, the drive member 5 and the coupling input member 6 jointly effect an axial coupling stroke along the length X. During this driving stroke movement or coupling displacement, the coupling input member 6 pushes the coupling interconnecting member 7 against the rebounding elastic force of the return member 10 in the forward drive direction V. During the course of the stroke movement, the engaging elements 6a and 8a move into engagement with one another, whilst the coupling interconnecting member 7 is simultaneously moved relative to the uncoupling member 11 until it is moved out of the rotationally fixed engagement with the uncoupling member 11. As this happens, the coupling interconnecting member 7 remains in a rotationally fixed engagement with the coupling sleeve 8. The coupling movement is restricted by means of a stop of the release element 16 on the coupling sleeve 8 and, in the embodiment illustrated as an example, on its proximal end face (FIG. 3).

FIG. 5 shows the injection device in the coupled state. The engaging elements 6a and 8a axially overlap so that the coupling engagement is established between the coupling input member 6 and the coupling sleeve 8, resulting in a rotationally fixed engagement. The engagement between the coupling interconnecting member 7 and the uncoupling member 11 is not released until after the coupling engagement has been reliably established.

To set a the dose, the user turns the dose-measuring member 18, which latches in easily releasable catch positions. The dose-measuring member 18 is connected to the coupling input member 6 in a rotationally fixed arrangement and is also unable to move axially, thus driving the latter with it in rotation. As a result of this dose-measuring movement of the coupling input member 6, the drive member 5, which is linearly guided in and opposite the forward drive direction V at 4b, moves in the proximal direction so that it then projects out from the housing part 4. The axial dose-measuring travel distance of the drive member 5 is the angle of rotation about which the dose-measuring member 18 is turned and the thread pitch in the thread engagement between the drive member 5 and the coupling input member 6, which moves into abutment against the coupling interconnecting member 7 in the forward drive direction V and against the housing part 4 in the direction opposite the forward drive direction V.

FIG. 6 shows the injection device with the container 2 still completely filled after a first dose has been set. In this state, the user pierces the skin with the injection needle for a subcutaneous injection. Once the injection needle has been positioned, the user operates the drive member 5 by pushing it into the housing part 4 in the forward drive direction V. During the first portion of the driving movement, i.e., the coupling movement or coupling stroke X, the drive member 5 drives the coupling input member 6 against the elastic rebound force of the return element 10 until the coupling engagement with the coupling sleeve 8 is established and the rotationally fixed engagement between the coupling interconnecting member 7 and the uncoupling member 11 is released. As soon as the coupling sleeve 8 and, hence jointly with it the coupling output member 9, is able to rotate freely about the common rotation axis R, the coupling stroke X is ended and is followed by a dispensing stroke constituting the second portion of the driving movement. During the dispensing stroke, the drive member 5 is pushed further in the forward drive direction V. Since the coupling input member 6 is not able to effect any further movement in the forward drive direction V beyond the axial abutment against the coupling interconnecting member 7, it turns in the thread engagement about the common thread axis R, being guided and rotationally fixed to the drive member 5. During its rotating motion in the coupling engagement, the coupling input member 6 drives the coupling sleeve 8, which in turn drives the coupling output member 9. The coupling sleeve 8 together with the coupling output member 9 is retained in the housing part 4 so that it can not be axially displaced. The rotating motion of the coupling output member 9 causes the forward driving motion via the thread engagement with the plunger rod 15 and its rotationally fixed linear guide at 4a, and the dispensing movement of the plunger rod 15 and, in conjunction with it, the plunger 3. As soon as the injection button 16 comes into abutting contact with the coupling sleeve 8 during the course of the driving and dispensing movement (FIG. 3), the dispensing procedure is terminated.

When the user releases the pressure on the release element 16, the return member 10 moves the coupling input member 6 via coupling interconnecting member 7 back into the retracted retaining position out of the coupling engagement, as illustrated in FIGS. 2 and 4. The coupling input member 6 and jointly the drive member 5, the dose-measuring member 18 and the dose display 20 are uncoupled by the disengaging movement of the coupling input member 6 from the coupling output member 9 and from the plunger rod 15. The plunger rod 15, on the other hand, is rotationally fixed to the housing part 4 again by means of the coupling interconnecting member 7 as it is retracted and by means of the and the uncoupling member 11.

FIG. 7 illustrates the injection device at the end of a final dispensing procedure, by means of which the container 2 was emptied.

In order to replace the empty container 2, the housing part 1 is released from the housing part 4, and in the embodiment illustrated as an example, by means of a screwing movement. When the housing parts 1 and 2 are released, the uncoupling member 11 is automatically moved relative to the housing part 4 in the direction opposite the coupling movement of the coupling input member 6, which is against the forward drive direction V in the embodiment illustrated as an example here. The housing part 4 provides a mount for the uncoupling member 11 accordingly. The axial distance travelled as a result by the uncoupling member 11 relative to the housing part 4 is as long as the coupling-stroke X, which means that once the housing parts 1 and 4 have been released, the uncoupling member 11 lies axially opposite the coupling input member 6 and blocks it and the coupling input member 6 can no longer be moved in the forward drive direction V, at least not into a coupling engagement with the coupling sleeve 8. The fact that the coupling input member 6 is blocked in the retracted position prevents the coupling output member 9 from moving into a rotationally fixed connection with the housing part 4 and thus prevents the plunger rod 15 from being pushed back. In other words, this ensures that the plunger rod can be pushed back into the housing part 4 without blocking.

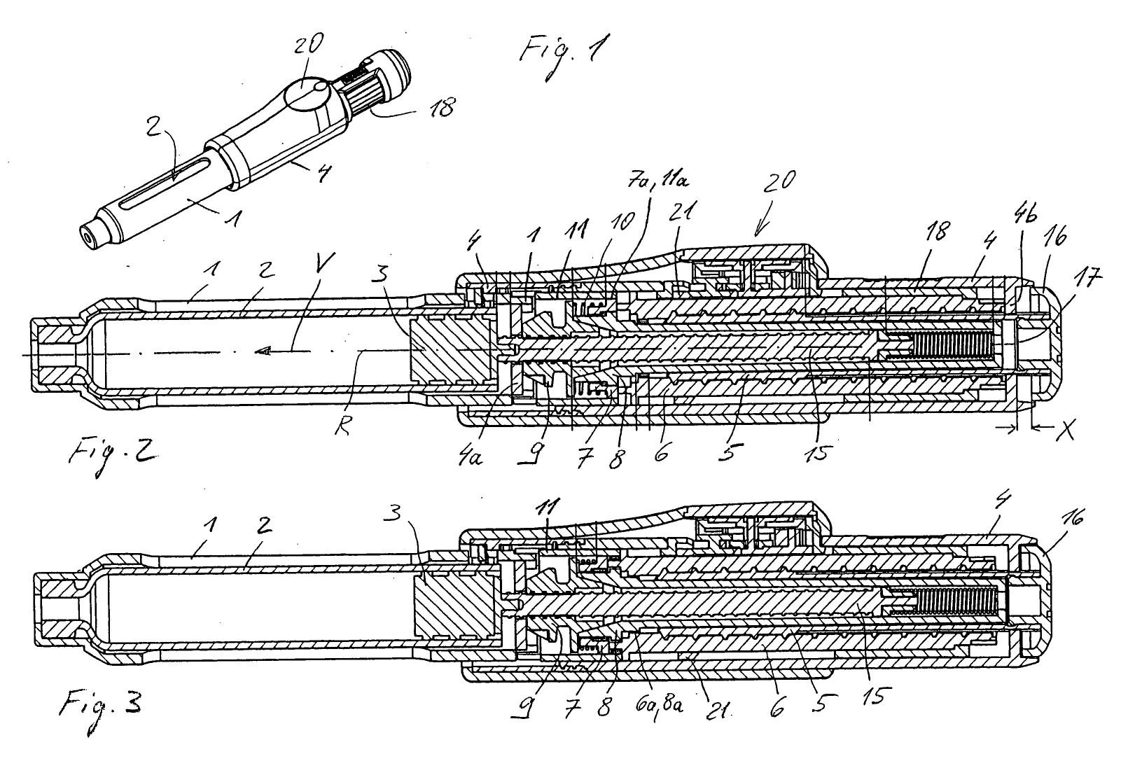

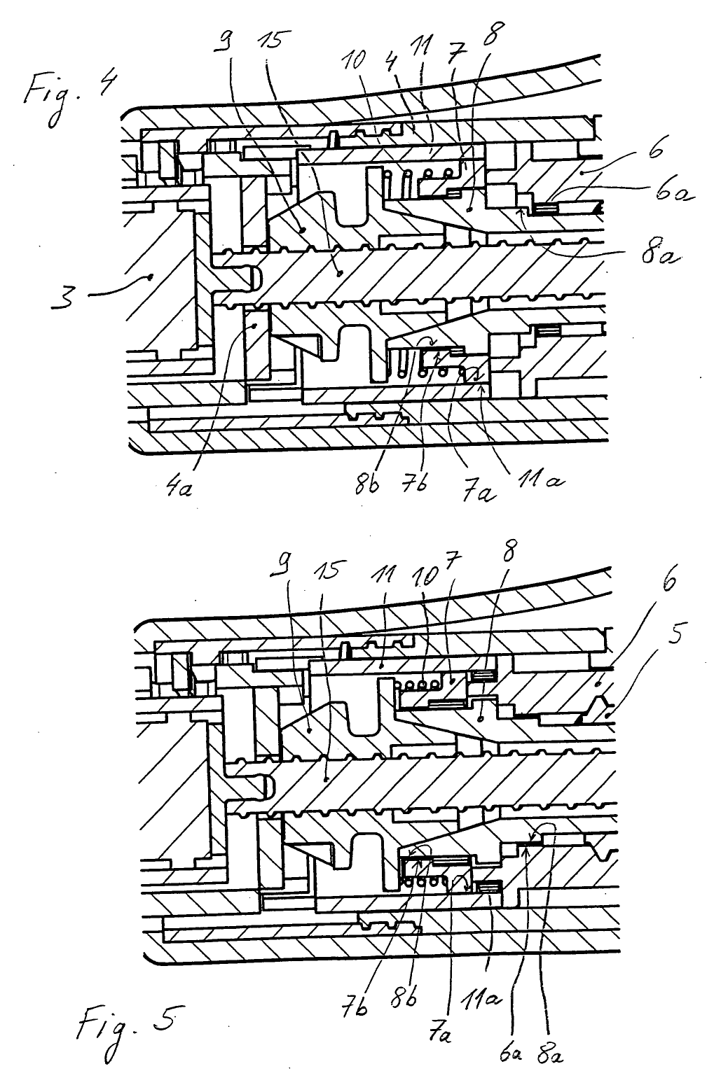

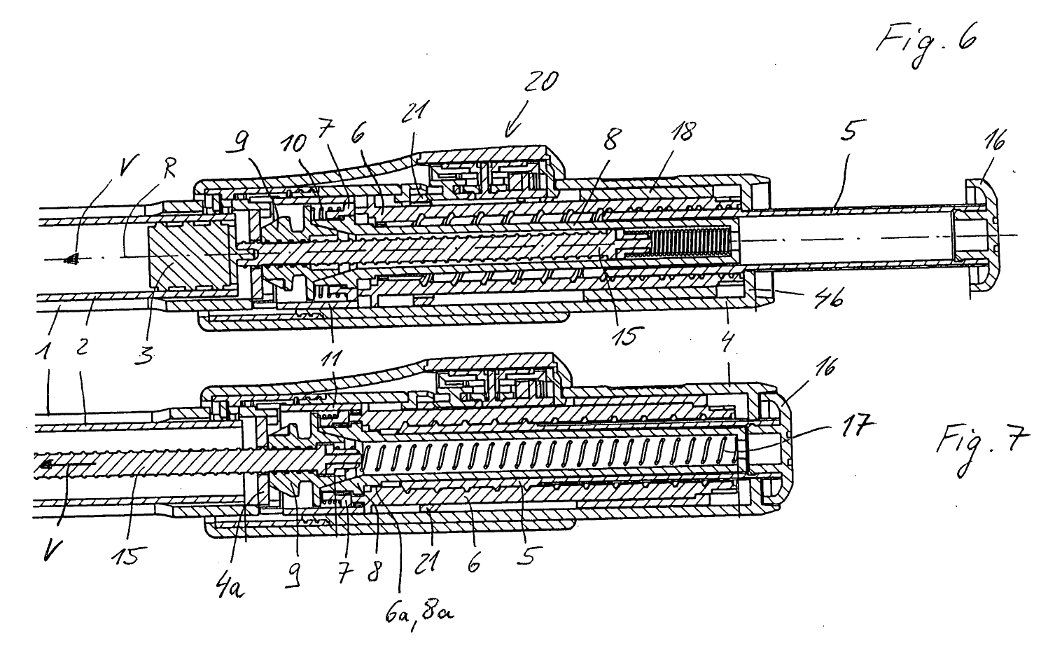

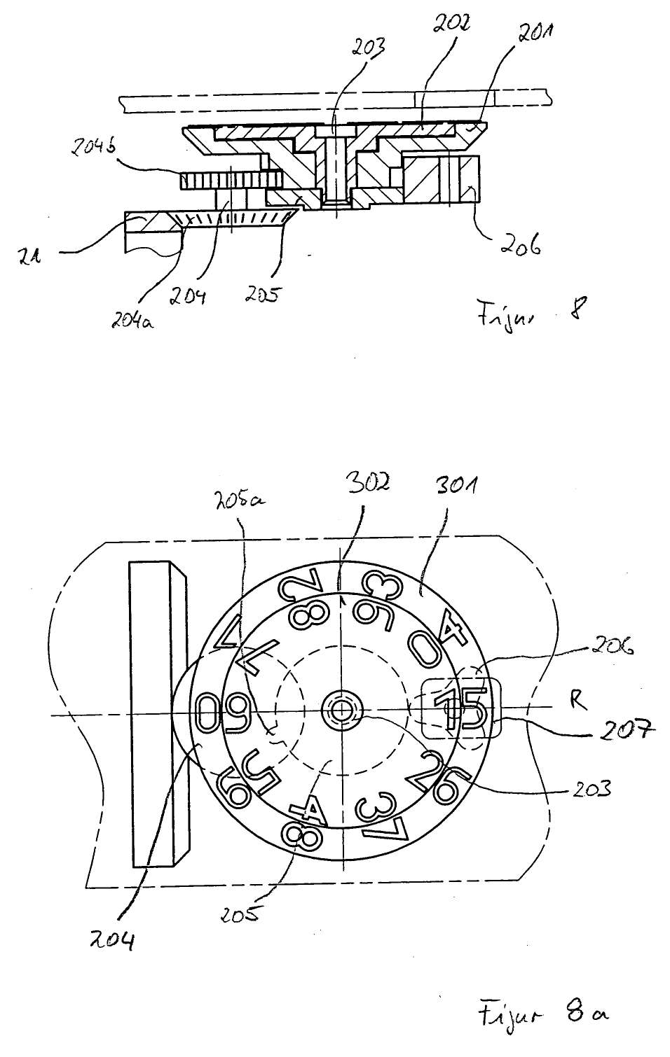

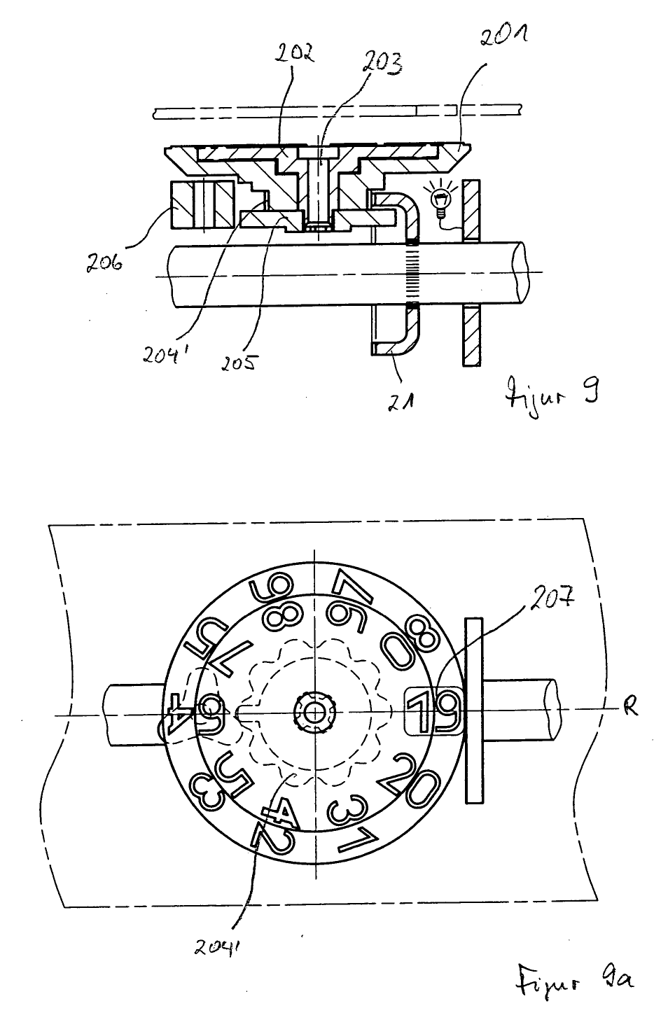

FIG. 8 shows a cross-section through a dose display proposed by the invention. Above the housing-side interface 21, a first display in the form of a counter dial 201 is driven by means of a gear 204a which is fixedly connected to a gear 204b or is made from a single part. Centrally mounted in this first display element 201 is another display element 202, which is rotatable relative to the first display element. Both display elements have scales 301, 302 incorporating numbers which may be applied with a fluorescent paint. Fixedly connected to the first display element 201 is a disc (205) with a radially projecting knob or a gear with only one tooth. It rotates with the display element 201 in the same direction and at the same speed and drives a capstan wheel 206 once with each rotation, which in turn is connected to the other display element 202 so that the rotation of the capstan wheel 206 turns the other display element 202 by another circle segment predefined by the geometry of this gear mechanism.

FIG. 8a illustrates a plan view of the described dose display.

FIG. 9 shows a dose display which differs from that illustrated in FIG. 8 in terms of the device-side drive. The rotating movement of the drive member 6 by means of which the dose-measuring element is always engaged as long is it is fitted on the device, is transmitted directly via a display coupling member 21 to the rotation axis of the first display element. To this end, the housing-side end of the first display element 201 is provided with teeth or the like, which mesh with the teeth of the display coupling member 21. In addition, an integrated light source is illustrated, which makes it easier to read the dose displayed at the window 207.

FIG. 9a shows a plan view of FIG. 9.

Embodiments of the present invention, including preferred embodiments, have been presented for the purpose of illustration and description. They are not intended to be exhaustive or to limit the invention to the precise forms and steps disclosed. Obvious modifications or variations are possible in light of the above teachings. The embodiments were chosen and described to provide the best illustration of the principals of the invention and its practical application, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. A device for administering measured doses of a liquid product with a dockable dose display, which device comprises
a) a housing with a reservoir for the product,
b) a plunger rod which can be displaced in order to convey the product in a forward drive direction,
c) a dose-measuring member which effects a dose-measuring movement in order to set a product dose to be administered,
d) a dose display with at least a first display element for displaying the set dose, and
e) a coupling which couples the dose display with the dose-measuring member in order to track and display the dose-measuring movement, wherein
f) the dose display has an interface extending sideways through the housing and coupled with the dose-measuring member, by which the display tracks and displays the dose measuring movement.

2. The device as claimed in claim 1, wherein the dose display can be detached at the interface.

3. The device as claimed in claim 1, wherein the interface is generally funnel-shaped to guide and center the dose display.

4. The device as claimed in claim 1, wherein a rotating motion of the dose-measuring member causes a rotating motion of the dose display by means of a gear mechanism.

5. The device as claimed in claim 1, wherein the dose display is always coupled with a drive member by means of the gear mechanism.

6. The device as claimed in claim 5, wherein a coupling member connects the dose-measuring member to the drive member during the dose-measuring movement.

7. The device as claimed in claims 6, wherein the coupling member releases the connection between the dose-measuring member and drive member during a first movement in the drive direction.

8. The device as claimed in claim 7, wherein the coupling member establishes a connection between the drive member and conveying mechanism during a further movement in the drive direction.

9. The device as claimed in claim 1, wherein both a setting at the dose-measuring member and the administering of the product causes a rotating movement of the at least one display element.

10. The device as claimed in claim 9, wherein the rotating motion effected during administering is opposite that generated by the dose-setting system.

11. The device as claimed in claim 5, wherein the rotation axis of the drive element of the at least one display element is disposed transversely to the rotation axis of the drive member.

12. The device as claimed in claim 5, wherein the drive member carries teeth for causing the at least one display element to be driven.

13. The device as claimed in claim 12, wherein the drive member drives the dose display both during measuring the dose and during dispensing.

14. The device as claimed in claim 12, wherein the rotating motion of the at least one display element of the dose display while measuring the dose is opposite that caused by the rotating motion caused by dispensing.

15. The device as claimed in claim 1, wherein the first display element centrally mounts another display element which is rotatable about the rotation axis of the dose display.

16. The device as claimed in claim 15, wherein the other display element is coupled with the first display element so that a complete rotation of the first display element causes a partial rotation of the other display element.

17. The device as claimed in claim 15, wherein each of the display elements is sub-divided into any but a mutually dependent number of circle segments.

18. The device as claimed in claim 1, further comprising a bushing disposed at the centre and extending through all the components connects the individual parts of the dose display to form a compact functional unit.

19. The device as claimed in claim 15, wherein the bottom end of the first display element is fixedly connected to a ring which has a driving element in the form of a cam on the side, which during a full rotation of the first display element further rotates the other display element by one circle segment by means of a capstan wheel connected to the other display element.

20. The device as claimed in claim 19, wherein the capstan wheel determines the circle segment about which the other display element is further rotated during a full rotation of the first display element.

21. The device as claimed in claim 20, wherein the other display element is further rotated by exactly 36°.

22. The device as claimed in claim 15, wherein the set dose can be read at a window.

23. The device as claimed in claim 22, wherein numbers are provided on the at least one display element in fluorescent paint.

24. The device as claimed in claim 22, wherein the at least one display element is made from a transparent material.

25. The device as claimed in claim 24, further comprising light sources mounted in the region of the window behind the at least one transparent display element.

26. The device as claimed in claim 25, wherein the light sources have different colors.

27. A dose display for coupling to an injection device or the like, comprising at least one display element, a gear mechanism and a central element which connects the display element, the gear mechanism and the injection device whereby the dose display can be coupled as a unit to appropriately prepared injection devices after the injection devices are manufactured.

28. The dose display according to claim 27, comprising two display elements.

29. The dose display according to claim 28, wherein the display elements are counter dials.
